(19) United States
(12) Patent Application Publication
(10) Pub. No.: US 2003/0015505 A1
(43) Pub. Date: Jan. 23, 2003

(54) Apparatus and method for sterilization of articles using capillary discharge atmospheric plasma

(75) Inventors: Dong Woo Yu, Demarest, NJ (US); Steven Kim, Harrington Park, NJ (US)

Correspondence Address: MORGAN LEWIS & BOCKIUS LLP, 1111 PENNSYLVANIA AVENUE NW, WASHINGTON, DC, 20004, US

(73) Assignee: SKION CORPORATION

(21) Appl. No.: 09/907,569

(22) Filed: Jul. 19, 2001

(51) Int. Cl. B23K 10/00

(52) U.S. Cl. 219/121.43; 219/121.58

(57) ABSTRACT

The present invention discloses an apparatus and method for sterilization of articles using capillary discharge atmospheric plasma. More specifically, an apparatus for sterilizing articles using substantially atmospheric pressure plasma includes a plasma generator generating the substantially atmospheric pressure plasma, wherein the plasma generator includes, first and second dielectrics facing into each other, wherein at least one capillary is formed in at least one of the dielectrics, and first and second electrodes on the first and second dielectric bodies, receiving the potential from the power supply, a processing chamber enclosing the plasma generator, and a power supply providing a potential to the plasma generator.

### BACKGROUND OF THE INVENTION

1. Field of the Invention

The invention relates to a plasma apparatus, and more specifically, an apparatus and method for sterilization of articles using capillary discharge atmospheric plasma (CDAP). Although the present application has a wide scope of applications, it is particularly suitable for effectively sterilizing contaminated medical instruments and any articles at a low cost by using high-density capillary discharge atmospheric plasma.

2. Discussion of the Related Art

A gas plasma sterilization process has been used in modern medical and dental practice. Although disposable articles are abundant in those industries, there are many reusable materials and articles that are required repeated sterilization. For example, there are surgical instruments and medical equipment that are employed in areas where sterilization is necessary, such as diagnostic equipment used in medicine.

Also, containers for holding medicines, vaccines, injectables, pills and the like, both in the manufacture, storage, and distribution of these products may fall into the above category. Further, it is applicable for articles for sterilization of clothing (e.g. fabric, paper and disposable), masks, eyeglasses and eyewear, gloves, shoes, and the like, and also the sterilization of sheets, bed-clothing, blankets and towels, used in operation areas and other areas of hospital, medical centers and treatment centers.

Conventionally, a number of approaches for performing sterilization have been employed, such as an ETO (ethylene oxide) method, a high-pressure steam autoclaving, and a conventional plasma treatment. For the ETO method, the major drawback is its dangerous toxicity. The autoclaving has also many restrictions in application when the process requires high temperature and is not suitable for materials that are affected by either moisture or high temperature.

Further, the conventional plasma treatment should be carried out under a sub-atmospheric condition, so that it has many limitations in size of articles. More importantly, since the system has to be maintained under a vacuum condition, it requires many electronics and peripheral components. In addition, efficiency of sterilization is not high enough to be used for industrial purposes.

### SUMMARY OF THE INVENTION

Accordingly, the present invention is directed to an apparatus and method for sterilization of articles using capillary discharge atmospheric plasma that substantially obviates one or more of problems due to limitations and disadvantages of the related art.

An object of the invention is to provide an apparatus and method for sterilization of articles using capillary discharge atmospheric plasma that effectively sterilizes contaminated articles including microorganisms as well as allows a low cost in maintenance.

Additional features and advantages of the invention will be set forth in the description that follows and in part will be apparent from the description, or may be learned by practice of the invention. The objectives and other advantages of the invention will be realized and attained by the structure particularly pointed out in the written description and claims hereof as well as the appended drawings.

To achieve these and other advantages and in accordance with the purpose of the present invention, as embodied and broadly described, an apparatus for sterilizing articles using substantially atmospheric pressure plasma includes a plasma generator generating the substantially atmospheric pressure plasma, wherein the plasma generator includes, first and second dielectrics facing into each other, wherein at least one capillary is formed in at least one of the dielectrics, and first and second electrodes on the first and second dielectric bodies, receiving the potential from the power supply, a processing chamber enclosing the plasma generator, and a power supply providing a potential to the plasma generator.

In another aspect of the present invention, an apparatus for sterilizing articles using substantially atmospheric pressure plasma includes a dielectric cylinder having at least one capillary formed therein and generating the substantially atmospheric pressure plasma out of the capillary, a power supply providing a potential, an article holder supporting the articles and receiving the potential from the power supply, wherein the article holder is surrounded by the dielectric cylinder, a metallic electrode receiving the potential; and a gas-tight chamber enclosing the dielectric cylinder, the article holder, and the metallic electrode.

In another aspect of the present invention, a method of sterilizing articles using substantially atmospheric pressure plasma includes the steps of placing the articles in the apparatus, wherein the apparatus includes a plasma generator having first and second dielectrics facing into each other, wherein at least one capillary is formed in at least one of the dielectrics, and first and second electrodes on the first and second dielectric bodies, applying a potential to the first and second electrodes; and generating capillary discharge plasma out of the capillary to sterilize microorganisms in the article.

In a further aspect of the present invention, a method of sterilizing articles using substantially atmospheric pressure plasma includes the steps of placing the articles in the apparatus, wherein the apparatus includes a dielectric cylinder having at least one capillary therein, an article holder supporting the articles, and surrounded by the dielectric cylinder, a metallic electrode coupled to the dielectric cylinder, and applying a potential to the metallic electrode and the article holder; and generating the substantially capillary discharge atmospheric pressure plasma out of the capillary to sterilize microorganisms in the articles.

It is to be understood that both the forgoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this application, illustrate embodiments of the invention and together with the description serve to explain the principle of the invention.

In the drawings:

FIG. 1 is a perspective view of an apparatus for sterilizing articles using atmospheric plasma according to a first embodiment of the present invention;

FIGS. 2A to 2H are cross-sectional views along with the line of II-II′ of the apparatus in FIG. 1;

FIG. 3 is a perspective view of an apparatus for sterilizing articles using atmospheric plasma according to a second embodiment of the present invention;

FIG. 4 is a perspective view of an apparatus for sterilizing articles using atmospheric pressure plasma according to a third embodiment of the present invention;

FIG. 5 is a partial detailed view and a cross-sectional view along with the line of V-V′ of the apparatus in FIG. 4;

FIG. 6 is a perspective view of an apparatus for sterilizing articles using atmospheric plasma according to a fourth embodiment of the present invention;

FIG. 7 is a cross-sectional view along with the line of VII-VII′ of the apparatus in FIG. 6;

FIG. 8 is a cross-sectional view of an apparatus for sterilizing articles using atmospheric plasma according to a fifth embodiment of the present invention; and

FIG. 9 is a cross-sectional view of an apparatus for sterilizing articles using atmospheric plasma according to a sixth embodiment of the present invention.

### DETAILED DESCRIPTION OF THE PREFERRED EMBODIMENTS

Reference will now be made in detail to the present preferred embodiments of the invention, examples of which are illustrated in the accompanying drawings. Whenever possible, the same reference numbers will be used throughout the drawings to refer to the same or like parts.

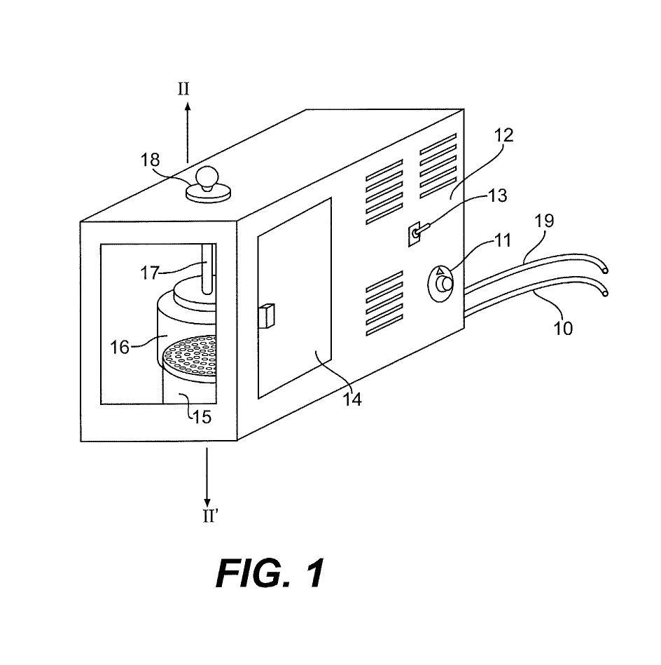

FIG. 1 illustrates a perspective view of an apparatus for sterilizing articles using atmospheric pressure plasma according to a first embodiment of the present invention.

As illustrated, the apparatus has a plasma generator 16 generating substantially atmospheric plasma between first and second dielectrics 15 and 20. At least one capillary 15′ is formed in at least one of the dielectrics 15 and 20. Articles such as medical instruments and tools to be sterilized are positioned between the first and second dielectrics 15 and 20.

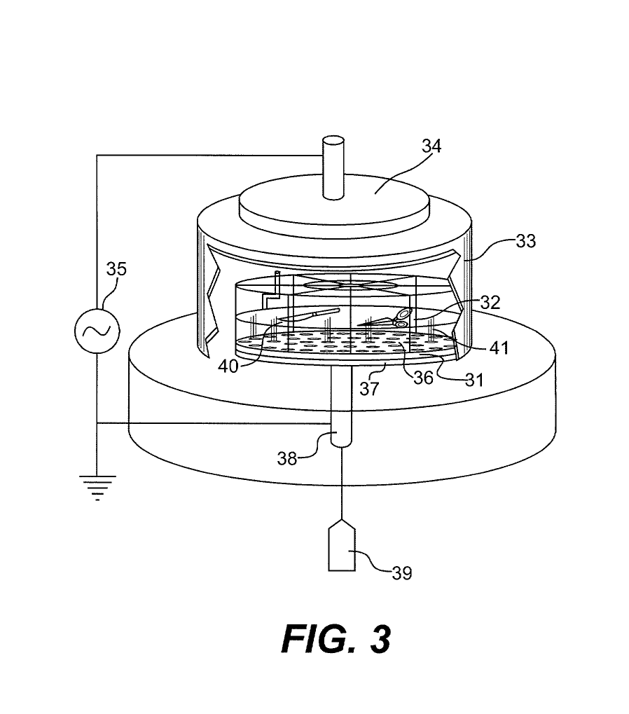

In order to process more than one articles, an article holder (shown in FIG. 3 as numeric reference 32) may be placed between the first and second dielectrics 15 and 20. Further, aldehyde vapors, such as formaldehyde and glutaraldehyde, may be added to the working gas in order to accelerate the above sterilization process.

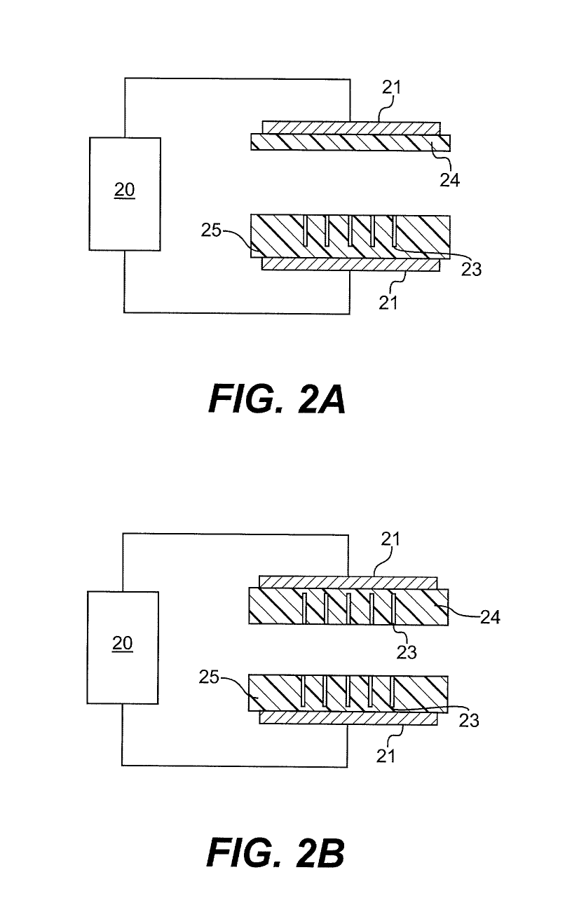

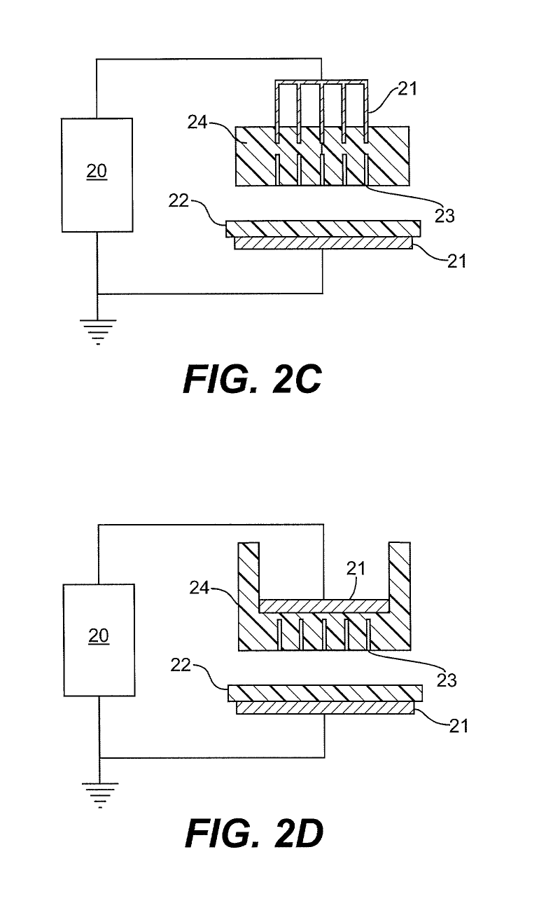

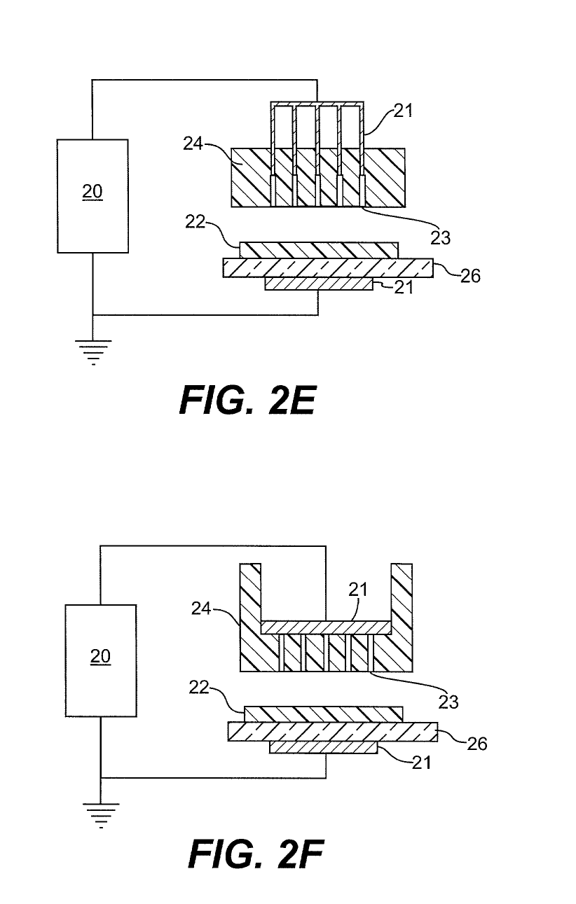

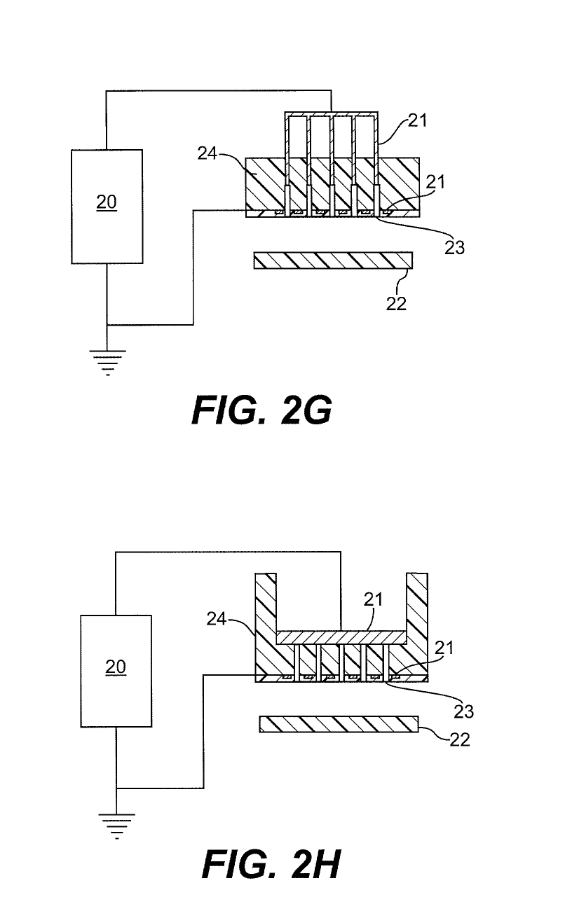

Gas tubes 10 and 19 are delivering a working gas provided by a gas supplier (not shown). The gas can be any kind of gases; preferably, it can be He, Ar, O₂, air, and any mixture of these gases. The plasma generator 16 is maintained under atmospheric pressure even if the working gases are circulating through the system. A power supply 12 is operated by a toggle switch 13 for turning on and off the power supply 12 and an adjustable knob 11 for adjusting an amount of potential to the plasma generator 16. The potential, preferably an AC potential, is applied through metallic electrodes (shown in FIGS. 2A to 2H) in the dielectrics 15 and 20. The applied potential varies with various conditions and is preferably in the range of 2 kHz to 200 MHz to generate substantially atmospheric plasma.

In order to receive articles into the apparatus, an article receiving mechanism, such as a supporter 17 and a handle 18 are arranged in the apparatus. By turning the handle 18, the supporter 18 makes the first dielectric 17 to move to an upward or downward direction, so that an article can be received to the plasma generator 16 through an entrance 14 of the apparatus.

FIGS. 2A to 2H illustrate examples of various designs for the plasma generator 16. As shown in FIGS. 2A and 2B, two dielectrics 24 and 25 are facing into each other. Metallic electrodes 21 are attached to each dielectric 24 and 25 for receiving a potential applied by a power supply 20. Preferably, one of the electrodes is grounded. At least one of the dielectrics 24 and 25 has at least one capillary 23 to generate high-density capillary discharged atmospheric plasma.

Optimum dimensions and the number of the capillaries may vary under operation conditions, such as a gas density and a process temperature, etc. For example, the number of capillaries may range from one to thousands. A thickness of the dielectrics 24 may be in the range of 2 mm to 30 mm. A diameter of each capillary is preferably in the range of 200 μm to 30 mm. These dimension and above-described operation conditions are also applicable for other designs of the plasma generators throughout the present invention, which will be discussed as follows:

In FIGS. 2C, 2E and 2G, an upper electrode on dielectric 24 has a pin shape. More specifically, tips of the pin electrodes 21 in FIG. 2C are not exposed to the capillary 23 formed in the dielectric 24, thereby suppressing arcing in sterilizing an electrically conductive article 22. Conversely, for an electrically non-conductive article 22 located on dielectric 26 in FIG. 2E, the pin electrodes 21 are exposed to the capillary 23 since there is no arcing problem for the nonconductive articles. In FIGS. 2G and 2H, a second electrode 21 is buried in the dielectric 24, SO that both conductive and nonconductive articles can be sterilized without any limitations.

FIG. 3 illustrates a perspective view of an apparatus for sterilizing articles using atmospheric pressure plasma according to a second embodiment of the present invention. The second embodiment is similar to the first embodiments.

Dielectric bodies 31 and 33 are spaced apart from each other. The bottom dielectric body 31 has at least one capillary therein, so that high-density capillary discharge plasma 41 is generated out of the capillary. A pair of electrodes 34 and 37 are attached to each dielectric body 31 and 33 receiving a potential from a power supply 35. A working gas is provided from the side of the bottom dielectric body 31. Further, as shown in the drawing, articles 40 are positioned in an article holder 32, so that a plurality of articles can be treated at the same time. As mentioned above, the above-discussed dimension and operation conditions are also applicable for this embodiment.

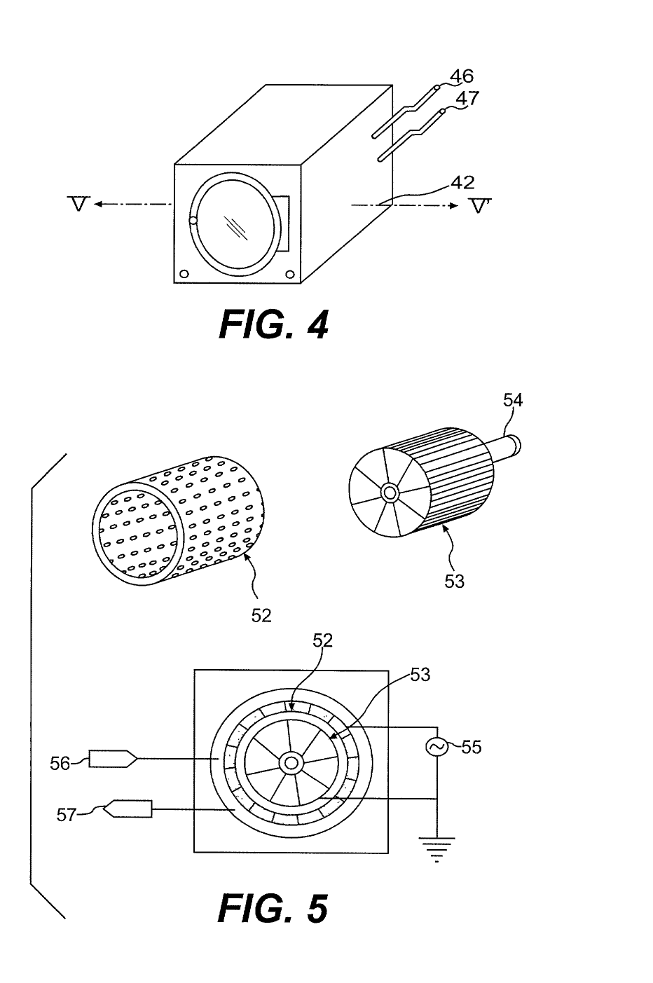

An apparatus for sterilizing articles using atmospheric plasma according to a third embodiment of the present invention is illustrated in FIG. 4. A detailed view in part and a cross-sectional view along with the line of V-V′ of FIG. 4 are shown in FIG. 5. In the third embodiment, a dielectric cylinder 52 having at least one capillary is located in the apparatus 41. An article holder 53 and an axis 54 rotate in operation to improve efficiency in sterilizing articles. Similarly, a working gas is provided through an inlet 46/56 and an outlet 47/57. Small size articles such as surgical tools or dental tools are effectively sterilized using this embodiment.

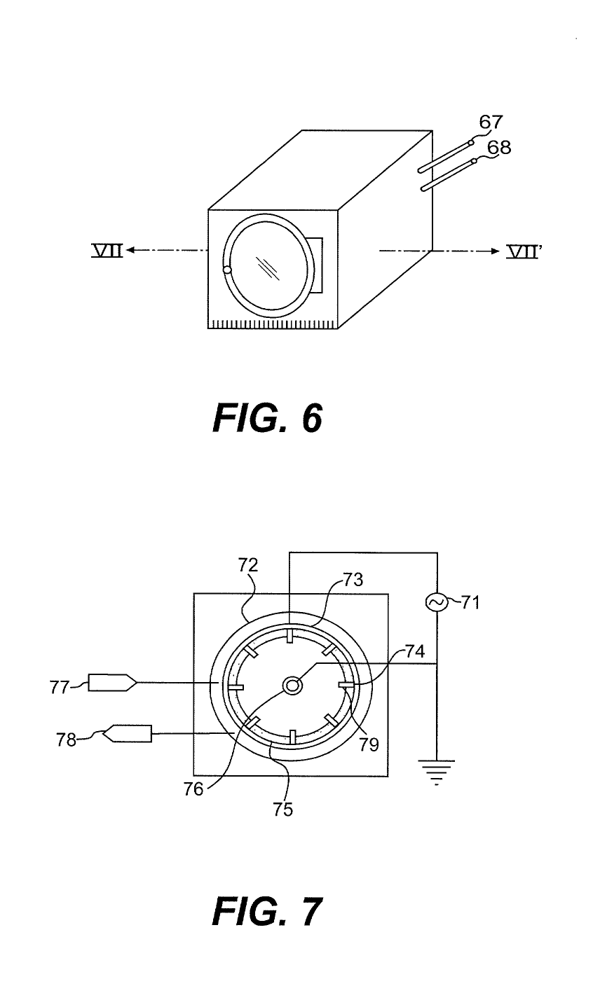

A fourth embodiment of an apparatus for sterilizing articles using atmospheric plasma shown in FIGS. 6 and 7 is similar to the third embodiment except for the motion of a dielectric cylinder 74 having at least one capillary 75. In this embodiment, the dielectric cylinder 74 rotates in operation, so that articles, such as fabrics, eyewear, gloves, shoes, containers, and suture materials, are properly sterilized. A potential is applied to a metallic cylinder surrounding the dielectric cylinder 74 and a central holder 76. The dielectric cylinder 74 has a concave portion 79 in order to facilitate treatment of articles in operation.

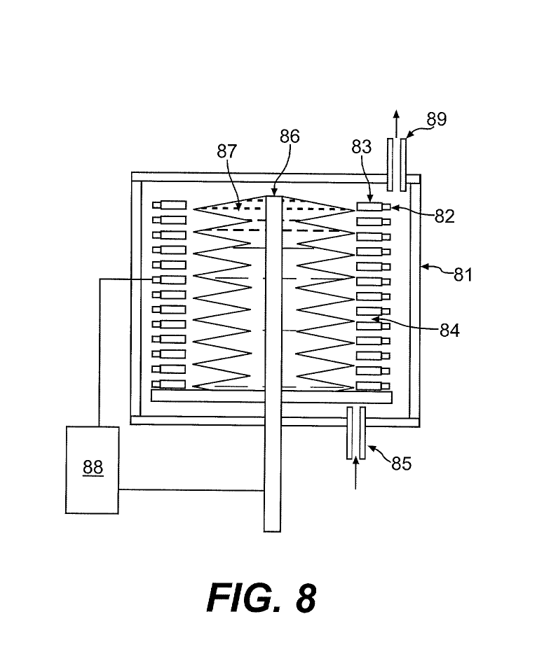

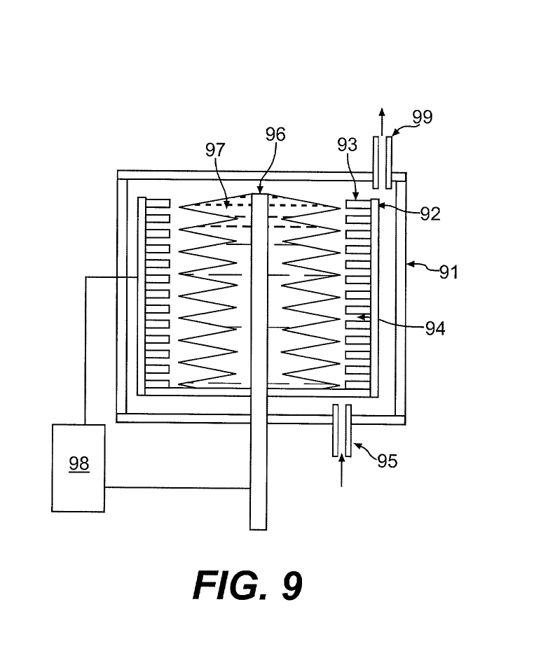

FIGS. 8 and 9 are cross-sectional views of an apparatus for sterilizing articles using atmospheric plasma according to sixth and seventh embodiments of the present invention. In these embodiments, dielectric bodies 83/93 and other elements of the apparatus are stationary in operation. The dielectric bodies 83/93 are cylindrical and have at least one capillary 84/94 therein. A center rod 86/96 act as electrodes, so that a potential is applied through 88/98. Similar to the previous embodiments, a gas inlet 85/95 and a gas outlet 89/99 are to provide a working gas into the chamber. A metallic cylinder 82/92 surrounding the dielectric bodies 83/93 may have a hole that substantially matches the capillary 84/94, as shown in FIG. 9. The metallic cylinder also acts as an electrode and may be grounded. Articles are positioned between the dielectric bodies 83/93, thereby being sterilized by high-density capillary discharge atmospheric plasma.

In order to demonstrate a feasibility of practical applications in industries, experiments were conducted using an apparatus and method as previously discussed in the present invention. As recommended in the procedure of the AOAC (Official Method Analysis of the Association of Official Analytical Chemists, 12^{th} ed., November 1975), Bacillus subtillis and Bacillius stearothermophilus were used in the experiments. Control spore strips (American Sterilizer Co. SPORIDI®) made of Bacillus subtillis and Bacillius stearothermophilus were tested under different conditions; ETOC (ethylene oxide certified) method, DHC (dry heat certified) method, and CDAP (capillary discharge atmospheric plasma) method.

First, the number of survivor for Bacillus subtillis after treated by the CDAP method was measured from 0 second to 120 seconds. Before the CDAP treatment, the number of survivor for Bacillus subtillis was about 950,000. The numbers were significantly reduced to about 600,000 in 60 seconds and about 200,000 in 120 seconds after the CDAP treatment.

D-value was also measured for the ETOC, DHC, and CDAP methods. D-value is described as the time necessary to reduce the population of cells by one log or 90%. These values are determined from plots of the number of survivors vs. time. Thus, based on the data, D-value is calculated for each method. For the ETOC and DHC methods, D-values were about 3.9 minutes and 1.5 minutes, respectively. D-value for the CDP method of the present invention was 2.95 minutes. The D-value of the present invention was higher than that of the DHC method. However, the DHC method has some disadvantages in application. For example, the DHC method cannot directly applied to a living human body or any animal because of hot and dry conditions. On the other hand, the CDAP treatment has almost no restriction in applying because its non-thermal nature of plasma.

Similar data were obtained for Bacillius stearothermophilus in the number of survivor and D-value. Before the CDP treatment, the number of survivor for Bacillus subtillis was about 4,200,000. The numbers were also significantly reduced to about 1,000,000 in 60 seconds and about 240,000 in 120 seconds after the CDAP treatment. For Bacillius stearothermophilus, D-value obtained for the sample treated by the CDAP method was lower than that by the DHC method. D-values for the CDAP and DHC method were about 1.54 and 1.90 minutes, respectively. Accordingly, the experimental results indicate that the CDAP method of the present invention is very effective in sterilizing Bacillius stearothermophilus.

As discussed above, the apparatus and method of sterilizing articles using capillary discharge atmospheric plasma can be utilized in any applications regardless of articles. Also, since no vacuum condition is required, cost of the apparatus and sterilization process is much reduced comparing to the conventional plasma methods. Further, in a sterilization process, the CDAP method of the present invention is more effective than the conventional sterilization methods, as demonstrated by the results of the experiments.

It will be apparent to those skilled in the art that various modifications and variations can be made in the apparatus and method for sterilization of medical instruments using atmospheric plasma of the present invention without departing from the spirit or scope of the inventions. Thus, it is intended that present invention covers the modifications and variations of this invention provided they come within the scope of the appended claims and their equivalents.

What is claimed is:

1. An apparatus for sterilizing articles using substantially atmospheric pressure plasma, comprising:
a plasma generator generating the substantially atmospheric pressure plasma, wherein the plasma generator includes, first and second dielectrics facing into each other, wherein at least one capillary is formed in at least one of the dielectrics, and first and second electrodes on the first and second dielectric bodies, receiving the potential from the power supply;
a processing chamber enclosing the plasma generator; and
a power supply providing a potential to the plasma generator.

2. The apparatus according to claim 1, further comprising an article receiving mechanism arranged to receive the articles into the processing chamber.

3. The apparatus according to claim 1, further comprising a gas supply for providing the plasma generating chamber with a working gas.

4. The apparatus according to claim 3, wherein the working gas includes one of Ar, He, O₂, air, and any mixture of Ar, He, O₂, air.

5. The apparatus according to claim 3, further comprising a sterilization enhancing gas.

6. The apparatus according to claim 5, wherein the sterilization enhancing gas is selected from the group consisting of aldehyde.

7. The apparatus according to claim 1, further comprising an article holder supporting the articles within the processing chamber.

8. The apparatus according to claim 1, wherein the articles include any reusable medical instruments, medical equipment, and dental tools.

9. The apparatus according to claim 1, wherein the articles are placed between the first and second dielectrics.

10. An apparatus for sterilizing articles using substantially atmospheric pressure plasma, comprising:
a dielectric cylinder having at least one capillary formed therein and generating the substantially atmospheric pressure plasma out of the capillary;
a power supply providing a potential;
an article holder supporting the articles and receiving the potential from the power supply, wherein the article holder is surrounded by the dielectric cylinder;
a metallic electrode receiving the potential; and
a gas-tight chamber enclosing the dielectric cylinder, the article holder, and the metallic electrode.

11. The apparatus according to claim 10, wherein the dielectric cylinder rotates in operation.

12. The apparatus according to claim 10, wherein the article holder rotates in operation.

13. The apparatus according to claim 10, further comprising a gas supply for providing a working gas.

14. The apparatus according to claim 10, wherein the working gas includes one of Ar, He, O₂, and air, and any mixture of Ar, He, O₂, and air.

15. The apparatus according to claim 13, further comprising a sterilization enhancing gas.

16. The apparatus according to claim 15, wherein the sterilization enhancing gas is selected from the group consisting of aldehyde.

17. The apparatus according to claim 10, wherein the articles include fabrics, eyewear, gloves, shoes, containers, and suture materials.

18. The apparatus according to claim 10, wherein an axis of the dielectric cylinder is substantially perpendicular to the ground level.

19. The apparatus according to claim 10, wherein an axis of the dielectric cylinder is substantially parallel to the ground level.

20. A method of sterilizing articles using substantially atmospheric pressure plasma, the method comprising the steps of:
placing the articles in the apparatus, wherein the apparatus includes,
a plasma generator having first and second dielectrics facing into each other, wherein at least one capillary is formed in at least one of the dielectrics, and first and second electrodes on the first and second dielectric bodies,
applying a potential to the first and second electrodes; and generating capillary discharge plasma out of the capillary to sterilize microorganisms in the article.

21. The method according to claim 20, wherein the articles includes any reusable medical instruments, medical equipment, and dental tools.

22. The method according to claim 20, further comprising the step of providing a working gas in close proximity to the articles.

23. The method according to claim 20, further comprising the step of simultaneously providing a sterilization enhancing gas with working gas.

24. The method according to claim 23, wherein the sterilization enhancing gas includes a gas selected from the group consisting of aldehyde.

25. A method of sterilizing articles using substantially atmospheric pressure plasma, the method comprising the steps of:
placing the articles in the apparatus, wherein the apparatus includes,
a dielectric cylinder having at least one capillary therein,
an article holder supporting the articles, and surrounded by the dielectric cylinder,
a metallic electrode coupled to the dielectric cylinder, and
applying a potential to the metallic electrode and the article holder; and
generating the substantially capillary discharge atmospheric pressure plasma out of the capillary to sterilize microorganisms in the articles.

26. The method according to claim 25, wherein the articles includes any reusable fabrics, eyewear, gloves, shoes, containers, and suture materials.

27. The method according to claim 25, further comprising the step of providing a working gas in close proximity to the articles.

28. The method according to claim 27, further comprising the step of simultaneously providing a sterilization enhancing gas with working gas.

29. The method according to claim 28, wherein the sterilization enhancing gas includes a gas selected from a group consisting of aldehyde.

30. The method according to claim 25, further comprising the step of rotating the article holder in operation of the apparatus.

31. The method according to claim 25, further comprising the step of rotating the dielectric cylinder in operation of the apparatus.
